(19) United States
(12) Patent Application Publication
(10) Pub. No.: US 2025/0380898 A1
(43) Pub. Date: Dec. 18, 2025

(54) SYSTEM AND METHOD FOR DISTINGUISHING SEIZURES UTILIZING HEART RATE AND AUTONOMIC BIOMARKERS

(71) Applicant: The Research Foundation for the State University of New York, Albany, NY (US)

(72) Inventors: David Auerbach, Syracuse, NY (US); Justin Ryan, Syracuse, NY (US); Kyle Wagner, Syracuse, NY (US)

(21) Appl. No.: 19/242,585

(22) Filed: Jun. 18, 2025

Related U.S. Application Data
(60) Provisional application No. 63/661,232, filed on Jun. 18, 2024.

(51) Int. Cl. A61B 5/00; A61B 5/024; A61B 5/0245; A61B 5/346; G16H 10/60; G16H 50/20

(52) U.S. Cl. CPC A61B 5/4094; A61B 5/02405; A61B 5/0245; A61B 5/346; G16H 10/60; G16H 50/20

(57) ABSTRACT

A system and method for distinguishing the type of seizures in a human patient, such as an epileptic seizure (ES), or a functional or dissociative seizure (FDS). The system and method use a diagnostic analytical platform that gets heart rate variability (HRV) analytical metrics from a ECG and uses an analytical diagnostic algorithm to determine if an ES or FDS has occurred in the patient. The diagnostic analytical platform can create a model for distinguishing that a predetermined type of seizure has occurred from the HRV analytical metrics.

### CROSS-REFERENCE TO RELATED APPLICATION

This invention claims the benefit of U.S. Provisional Patent Application No. 63/661,232, filed on Jun. 18, 2024, the entirety of which is hereby incorporated herein by this reference.

### BACKGROUND OF THE INVENTION

### 1. Field of the Invention

The present invention generally relates to medical devices. More particularly, the present invention relates to a system and method for distinguishing between convulsive epileptic seizures from functional or dissociative seizures.

### 2. Description of the Related Art

Epileptic seizures (ES) are due to transient electrical disturbances in neurons that lead to synchronous discharges. Functional or dissociative seizures (FDS) result in motor, sensory, and behavioral manifestations that are similar to ES, but there are no abnormalities detected on the electroencephalogram (EEG). FDS are associated with a history of psychological distress, particularly in individuals with a history of trauma/abuse. FDS occur in 20-40% of patients admitted to the epilepsy monitoring unit, 40% of patients in general neurology clinics, and ranks in the top 3 neuropsychiatric diseases (3:100,000 patients/year). The risks, treatments, and prognosis differ for ES versus FDS. In addition to people with drug-resistant ES being at a high risk of sudden death, there is a 2.5-fold higher rate of sudden death in individuals with FDS, compared to the general population.

Admission to the epilepsy monitoring unit (EMU) for video/EEG/ECG is the current standard ES vs. FDS diagnostic test. As EMUs are primarily only available in major cities, they are not accessible to many affected populations worldwide. EMU admissions are burdensome, costly, and can sometimes yield inconclusive results (e.g., habitual events not recorded during admission). Among patients admitted to the emergency department and initially diagnosed as ES, 26.5% are in fact FDS. This causes delays in FDS diagnosis and treatment (e.g., cognitive behavior therapy). Misdiagnosis results in unnecessary medications and inappropriate medical treatments/procedures (e.g., high-dose benzodiazepine administration resulting in intubation).

Patients with a history of ES and FDS exhibit chronic and acute seizure-related autonomic disturbances. This is particularly evident in people with refractory epilepsy and for SUDEP cases. There are conflicting study results as to whether autonomic function differs in people with ES vs. FDS, and surrounding ES vs. FDS. This is likely due to analysis during heterogenous timepoints and recordings of variable durations, as well as non-standardized methodologies.

Accordingly, there is a need for an alternative, non-invasive ES vs. FDS diagnostic system that is available to patients outside the hospital or in geographic areas with limited access to healthcare. The present invention is primarily directed to improving access to ES vs. FDS diagnostics.

### BRIEF SUMMARY OF THE INVENTION

Briefly described, the present system and method distinguish ES from FDS events by evaluating the temporal evolution of autonomic function throughout a series of seizures. The present system utilizes heart rate (HR)/heart rate variable (HRV)-based models for diagnostic purposes. Due to the expansion of wearable ECG recorders, the HRV-based diagnostic biomarker model invention provides an out-of-hospital tool to improve access to care.

The autonomic nervous system connects the brain and heart and regulates cardiac electrical activity. Consequently, quantification of cardiac beat-to-beat variability (heart rate variability, HRV) is a validated non-invasive method for assessing total autonomic (SDNN & Total Power), parasympathetic (RMSSD, HF power), baroreflex (LF power), and sympatho-vagal autonomic function (LF/HF). All of these parameters are utilized in the present system to distinguish ES v FDS.

In an embodiment, the invention provides a system for distinguishing the type of seizures in a human patient and includes an electrocardiogram recording device (ECG) and an HRV-based algorithm. The diagnostic analytical platform is uses the cardiac ECG, HRV analytical metrics, and an analytical diagnostic algorithm to distinguish epileptic seizure (ES) vs. functional or dissociative seizure (FDS).

In one embodiment, the invention provides a method for distinguishing the type of seizures in a human patient. An electrocardiogram (ECG) is used to acquire recordings. HRV-analysis is performed on the recordings throughout or during specific timepoints during the 2.5-hours leading up to and 2.5-hours following the ES or FDS event. The change in peri-ictal HRV metrics from baseline (>60-minutes prior to the seizure) are input into an analytical diagnostic model (i.e., logistic regression, but could also other models could be used) that distinguishes whether the event was ES or FDS.

In another embodiment, the invention provides a device for distinguishing the type of seizures that have occurred in a human patient, which has an electrocardiogram (ECG) selectively recording cardiac data from a patient and providing heart rate variability (HRV), and a diagnostic analytical platform that is configured to distinguish that a predetermined type of seizure has occurred in the patient based upon received cardiac data from the ECG and brain activity data from the EEG.

Accordingly, the present invention provides an advantage in diagnosing ES from FDC with less specialized and inexpensive existing medical devices, which can also be used in non-hospital settings. The present invention also has industrial applicability in the manufacture of medical equipment and diagnostic systems. Other objects, features, and advantages of the present application will be apparent to one of skill in the art after review of the present application.

### BRIEF DESCRIPTION OF THE DRAWINGS

FIG. 1 is a diagram of one embodiment of the present system for distinguishing types of seizure in a patent.

FIG. 2 is a series of graphs illustrating the temporal evolution of HR and autonomic function data surrounding a seizure event.

FIG. 3A is a graph of convulsive ES group averages during every post-ictal epoch show an increase in heart rate and reductions in time domain and frequency domain.

FIG. 3B is a graph of convulsive ES group averages during every post-ictal epoch with autonomic measures compared to show an increase in heart rate and reductions in time domain and to FDS.

FIG. 4 is a series of graphs of pre- and post-ictal coefficient of variation (100×standard deviation/mean).

FIG. 5 is a series of graphs of the temporal dependence of heart rate and HRV in distinguishing convulsive ES vs. convulsive FDS.

### DETAILED DESCRIPTION OF THE INVENTION

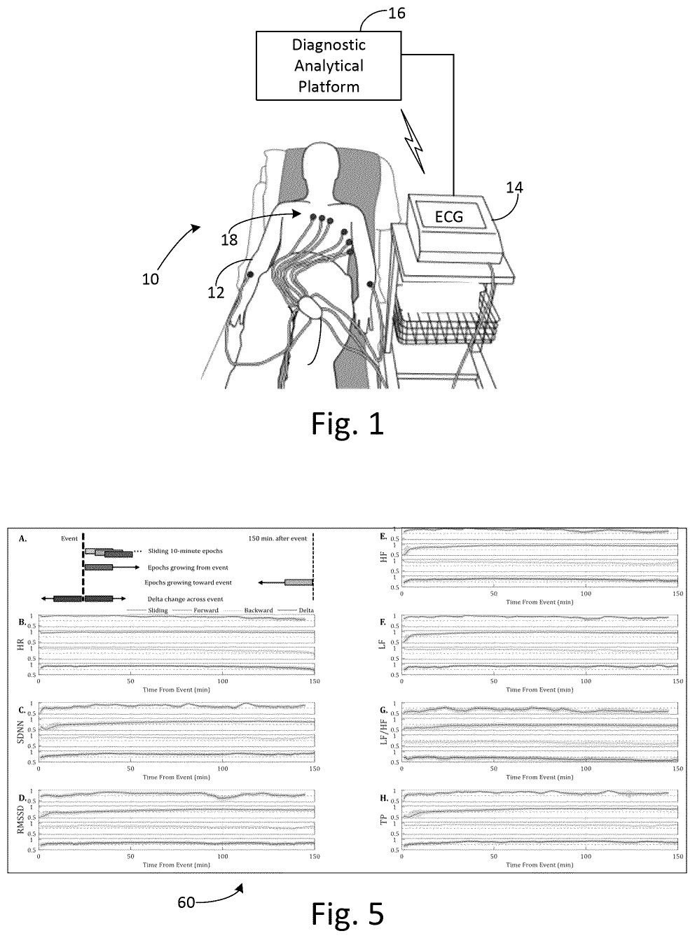

With reference to the figures in which like numerals represent like elements throughout the several views, FIG. 1 is a diagram of one embodiment of the present system 10 for distinguishing types of seizure in a patent 12. In this embodiment, the system 10 includes an electrocardiogram device (ECG) 14 selectively recording and transmitting cardiac data from the patient 12, and a diagnostic analytical platform 16 is communicatively connected to the ECG 14 and receives, at least, heart rate variability (HRV) data therefrom. The communication be wired or wireless as shown in FIG. 1. Furthermore, the ECG 14 and diagnostic analytical platform 16 can all be physically resident in the same device or can be separately located from each other.

The ECG 14 has leads 18 connected to the patient 12 to gather cardiac data. The diagnostic analytical platform 16 is configured to distinguish that a predetermined type of seizure occurred in the patient 12 based upon predetermined changes being present or absent from the HRV analytical data. The diagnostic analytical platform 16 is configured to utilize an analytical diagnostic algorithm on the HRV analytical data to distinguish that a predetermined type of seizure has occurred in the patient 12.

As is further described herein, the predetermined type of seizure is one of: an epileptic seizure (ES), or a functional or dissociative seizure (FDS). An ES onset can be distinguished by the first appearance of peri- or post-ictal changes in the HRV analytical data, as is further described herein. And an FDS onset can be distinguished by the presence or absence of changes in HRV analytical data received from the ECG 14.

In one embodiment, the present system 10, the electrocardiogram (ECG) 14 is used to acquire recordings, and HRV-analysis is performed by the diagnostic analytical platform 16, or at the ECG 14 itself, on the recordings throughout or during specific timepoints, such as during the 2.5-hours leading up to and 2.5-hours following the ES or FDS event. The change in peri-ictal HRV metrics from baseline (>60-minutes prior to the seizure) are input into an analytical diagnostic model at the diagnostic analytical platform, such models as a logistic regression, tree-based methods, neural networks and support-vector machines, K-nearest neighbor, and other traditional statistical methods, that are able to distinguish whether the event was ES or FDS.

The diagnostic analytical platform 16 can be further configured to distinguish predetermined types of seizures in a series of seizures within a predetermined duration within the patient 12 such as a periods of hourly observation of the patient 12. Further, the computer platform 16 can be configured to create a model for distinguishing the onset of a predetermined type of seizure in the patient 12 based upon received HRV analytical data from the ECG 14. In an embodiment, the created model can be a logistic regression model, as shown below.

To demonstrate the efficacy of the present system 10, a study included consecutive patients (18-65 years, n=99) with an ES or FDS recorded while in the University of Rochester EMU (2018-2021). To generate a homogeneous cohort that was free of potential clinical confounders, we excluded participants with comorbidities, medications, or devices that interfere with cardiac and autonomic function (cardiovascular or pulmonary disease, diabetes, smoking, stimulants, beta-adrenergic blockers, neural or cardiac devices).

Continuous time-synchronized video-audio-EEG-ECG 14 recordings were acquired and annotated during the entire EMU admission, and stored using the Natus-XItek. Video/EEG data was analyzed using Persyst software and correlated with the patient's physical manifestations. At least two neurologists/epileptologists classified the event as ES or FDS, and identified the onset and the end of the event based on the video/EEG evidence. ES onset was defined as the first appearance of ictal changes in the EEG (i.e., diffuse desynchronization, focal or generalized epileptiform discharge). In the uncommon instance that an obvious clinical manifestation (i.e., staring, unresponsiveness, automatisms) preceded electrographic changes, the clinical onset was used. The end of an ES was defined as cessation of ictal EEG discharges. For FDS, the onset was defined as the first appearance of the typical symptoms of the attack (e.g., falling, unresponsiveness). When the FDS onset was more gradual, we defined the onset as when the behavioral changes became most prominent (e.g., extreme agitation, hyper-motor activity). The end of the FDS was when the person regained control and resumed normal behavior. If the participant had multiple FDS that were typical, we selected the event that had the most distinct onset/offset.

The changes in autonomic function surrounding ES and FDS events were categorized in two groups: convulsive (those that manifested a significant component of physical/muscle exertion that would likely increase metabolic rate and thus heart rate) and non-convulsive (events without a significant motor component). Our classification of convulsive and non-convulsive semiologies for ES uses ILAE terminology, and FDS terminology is similar to that outlined in Asadi-Pooya (2019), which captures the vast types of FDS semiologies. Non-convulsive ES event semiology included automatisms, atonic, focal myoclonic, focal tonic, autonomic, behavior arrest, cognitive, emotional, sensory, and absence seizures. Non-convulsive FDS event semiology included semiology similar to non-convulsive ES as well as minor distal limb tremors, and blinking/eyelid flutters. Convulsive ES event semiology included generalized onset seizures that were tonic-clonic, clonic, tonic, or myoclonic-tonic-clonic, as well as focal onset seizures that were focal to bilateral tonic-clonic, or focal onset hyperkinetic seizures (e.g. frontal lobe seizures). Convulsive FDS event semiology included tonic, clonic or dystonic, generalized movements (excluding minor tremor), rigor-like movements, pelvic thrusting, pedaling and side-to-side movements of the head and body.

The Cardiac ECG 14 analysis utilized an M12 Holter System software (Global Instrumentation, USA) was used for automatic QRS detection and beats were annotated as normal/sinus or non-sinus/artifact, then manually adjudicated. To remove the effect that abnormal (non-sinus, ectopic, or aberrant) beats have on HRV measurements, each epoch needed to have >80% sinus beats. In fact, 92% of all epochs used in analysis had >95% sinus beats. The few ectopic or anomalous beats were removed and the appropriate RR interval in the cycle was interpolated. This is critical for spectral frequency methods, where interpolation and resampling facilitate maintaining the phase and cycle of RR interval oscillations, assuring the accurate calculation of the HRV frequency domain measures. All HRV analysis was performed according to the recommendation of the European Society of Cardiology Task Force.

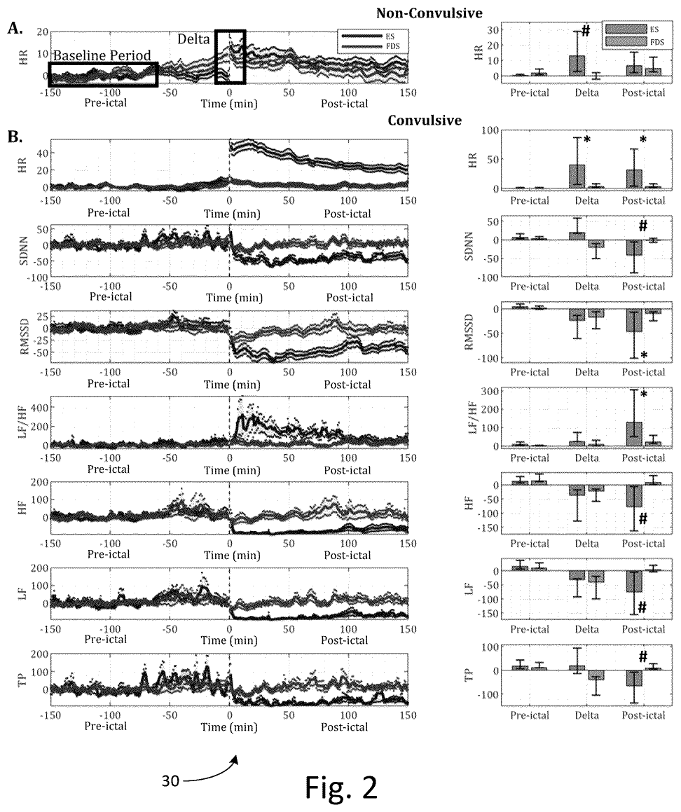

FIG. 2 is a series of graphs 30 illustrating the temporal evolution of HR and autonomic function data surrounding a seizure event. FIG. 2, Graph (A) shows timepoints used for analysis. Pre-ictal (150 min leading up to event onset), post-ictal (150 min following event end), and Delta (first 6 min post-ictal minus last 6 min pre-ictal) are all computed as percent change from baseline (150-60 min pre-ictal). FIG. 2, graph A, shows temporal evolution of HR in. patients with non-convulsive events. Bar plots represent the average pre-ictal, Delta, and post-ictal percent change from baseline. ES patients exhibited significantly larger Delta than FDS. FIG. 2, graph B shows temporal evolution of heart rate and HRV measures surrounding convulsive events. ES patients exhibited a sustained increased in heart rate and decrease in autonomic function. Statistical significance: * p<0.05 in Wilcoxon's rank-sum test; #p<0.05 in Wilcoxon's rank-sum test and logistic regression adjusted for age, sex, and administration of lorazepam. All HRV abbreviations and autonomic correlates are defined in Table 1 below.

TABLE 1
Standard definitions and autonomic correlates
of heart rate variability measures.

| | Abbrevi- | | Autonomic |
|---|---|---|---|
| Domain | ation | Definition | Correlation |
| Time | SDNN | Standard Deviation of | Total Autonomic |
| | | Normal-Normal (NN) | Function |
| | | Intervals | |
| Time | RMSSD | Root Mean Square of | Vagal |
| | | Successive NN Intervals | (Parasympathetic) |
| | | | Function |
| Frequency | HF | High Frequency Power | Vagal |
| | | (0.15-0.4 Hz) | (Parasympathetic) |
| | | | Function |
| Frequency | LF | Low Frequency Power | Baroreflex |
| | | (0.04-0.15 Hz) | Activity |
| Frequency | TP | Total Power (≤0.4 Hz) | Total Autonomic |
| | | | Function |
| Frequency | LF/HF | Ratio of LF divided by HF | Sympatho-vagal |
| | | | balance |

While some patient participants had multiple seizure events during admission, to avoid over-weighting a single individual, for each participant we selected one event that was most representative of the person's habitual semiology. To study the temporal evolution of HR and autonomic function surrounding an event, for each participant we analyzed the 2.5 h seizure-free pre-ictal period, and the 2.5 h seizure-free post-ictal period (FIG. 2, graph A). To evaluate the temporal changes in autonomic function, we performed HRV analysis using 5-minute epochs with 1-minute sliding windows. Ictal HRV analyses were not included as ECG recordings during the ictal period did not meet the criteria for time domain HRV analysis in 63% of the study cohort (i.e., <1-minute, muscle artifact). The ECG analyst was blinded to the ES/FDS diagnosis.

As there may be inter-patient differences in baseline autonomic function, we evaluated the percent change within each individual by normalizing all values to a baseline period 60-150 min preceding the seizure onset. FIG. 2, graph B. The pre-to-post-ictal change (delta) was measured by subtracting the average of the last 2 pre-ictal epochs from the average of the first 2 post-ictal epochs. As clinical semiology may impact heart rate and autonomic measures, convulsive and non-convulsive events were analyzed separately.

Nonparametric tests and adjusted regression models were used to test for differences between ES vs. FDS. Wilcoxon's rank-sum test was applied for continuous values. Pearson's chi-squared test was used for dichotomized variables. For each ECG 14 measure and time period, logistic regression with the ES/FDS diagnosis as the outcome was performed to calculate the adjusted association, which included the age at recording and sex. Post-ictal and delta measures included adjustments for lorazepam administration. Uni-variable and multi-variable logistic regression models evaluated the sensitivity/specificity of HRV measures to distinguish ES vs. FDS and develop receiver operating characteristic (ROC) curves. All data processing and analytics were performed using MATLAB 2022 (MathWorks, Massachusetts). Statistical significance was defined as p<0.05, and Bonferroni corrections were applied for comparisons with multiple timepoints.

Logistic regression models were trained over 1000 iterations using randomly generated 75%-training 25%-testing groups, and 95% confidence intervals for sensitivity, specificity, and ROC curves were computed for each HRV measure and timepoint. In each iteration, sensitivity and specificity were calculated based on the predicted ES or FDS label using the randomly selected testing cohort. One label was assigned per individual per iteration. To avoid overfitting the models, the feature space was limited to at most 3 HRV measures per model, and did not include age, sex, or lorazepam. Models with ROC≥0.7 are reported in Table 2 below.

For univariate logistic regression models, optimal cut-off thresholds were determined from the ROC curves. Using MATLAB's perfcurve function, the optimal threshold is calculated using a slope method that maximizes both sensitivity (true ES predictions) and specificity (true FDS predictions. Using the inverse logit function, the threshold corresponding to the optimal ROC point was used to determine the optimal percent-change cut-off for each ECG measure to distinguish ES vs. FDS events.

There were 187 consecutive participants free of confounding factors were consented. 88 were excluded due to insufficient data or poor signal quality (n=16), no events recorded or inconclusive diagnostic results (n=29), non-ES/FDS events (n=6), non-typical events (n=30), dual ES/FDS diagnosis (n=1), or did not meet the selection criteria (n=6). Thus, the study includes 53 ES and 46 FDS participants with a diagnostic habitual event recorded in the EMU (Table 2). As FDS are more common in women and people 15-35 years of age [1,2], there are significant differences between groups in terms of age, sex, percentage of convulsive seizures, and percentage of participants that received lorazepam. These variables are adjusted for in the logistic regression models.

Table 2 illustrating the Clinical Characteristics of the study. Categorical variables were compared using Pearson's Chi-squared test. Continuous variables were compared using Wilcoxon's rank-sum test. IQR=Inter-quartile range; SSRI=selective serotonin reuptake inhibitor; MAOI=Monoamine oxidase inhibitors; SNRI=serotonin and norepinephrine reuptake inhibitors.

TABLE 1

| Characteristic | ES | | FDS | | p-value |
|---|---|---|---|---|---|
| N | 53 | | 46 | | 0.320 |
| Convulsive Event (%) | 17 | (32) | 25 | (54) | 0.025 |
| Female (%) | 32 | (60) | 37 | (80) | 0.030 |
| Age, years median (IQR) | 38 | (26.75, 49.5) | 30 | (23, 38) | 0.012 |
| Patient Received Lorazepam | 15 | (28) | 5 | (11) | 0.031 |
| (%) | | | | | |
| Awake (vs Asleep) at Event | 32 | (60) | 45 | (98) | <0.001 |
| (%) | | | | | |
| Event Duration, seconds | 96 | (55.25, 148.5) | 292 | (92, 736) | <0.001 |
| median (IQR) | | | | | |
| Sleep Apnea (%) | 4 | (8) | 2 | (4) | 0.506 |
| Arrythmia (%) | 1 | (2) | 1 | (2) | 0.919 |
| Anxiety (%) | 21 | (40) | 32 | (70) | 0.003 |
| Depression (%) | 17 | (32) | 29 | (63) | 0.002 |
| Bipolar (%) | 3 | (6) | 6 | (13) | 0.202 |
| Anti-Seizure Medications | 51 | (96) | 28 | (61) | <0.001 |
| (ASM) at Admission (%) | | | | | |
| Na+ Channel Blocker (%) | 38 | (72) | 18 | (39) | 0.001 |
| GABA Modifier/ | 13 | (25) | 7 | (15) | 0.250 |
| Benzodiazepines (%) | | | | | |
| SV2A Receptor Blocker (%) | 30 | (57) | 16 | (35) | 0.030 |
| Anti-Depressant (AD) | 15 | (28) | 19 | (41) | 0.174 |
| Medications at Admission (%) | | | | | |
| Tricyclic AD (%) | 0 | (0) | 2 | (4) | 0.125 |
| SSRI, MAOI, SNRI AD (%) | 15 | (28) | 17 | (37) | 0.358 |
| History of Physical, Verbal, | 7 | (13) | 17 | (37) | 0.006 |
| Emotional, Sexual Abuse (%) | | | | | |

FIG. 2 illustrates the relative change in HR in the 150 min leading up to and 150 min following ES and FDS. The pre-to-post-ictal change in HR (delta) is significantly larger for ES vs. FDS, both for non-convulsive and convulsive events, analyzed separately. The post-ictal increase in HR is significantly larger for convulsive ES vs. convulsive FDS. Adjusted logistic regression models indicate that non-convulsive HR-delta is significantly different in ES vs. FDS when adjusted for age, sex, and lorazepam administration. High resolution analysis (1-minute epochs, 10-second steps) closely surrounding the seizure confirms these results.

The change in HR closely surrounding the event was evaluated (10-60 s pre-/post-ictal). Compared to baseline, there is a significant increase in pre-ictal and post-ictal HR for FDS events, and post-ictal HR for ES. There is a significantly larger increase in HR and prevalence of clinical tachycardia (HR>100 bpm) during the post-ictal period for convulsive ES vs. convulsive FDS.

For examination of the fluctuations in HR, we evaluated the changes in autonomic function surrounding the seizures. For ES participants, post-ictal HRV measures are significantly different than baseline timepoints. Despite psychological and anxiety changes often preceding FDS, HR and all HRV measures are surprisingly similar during the baseline and all peri-ictal timepoints (pre-ictal, pre-/post-ictal delta, and post-ictal periods).

While there are no differences in HRV measures for non-convulsive ES vs. non-convulsive FDS, autonomic function differs during the post-ictal period following convulsive ES vs. convulsive FDS (FIG. 2, graph B). Total autonomic (SDNN & TP), vagal (RMSSD, HF), and baroreflex function (LF) are lower following convulsive ES vs. convulsive FDS, which indicates post-ictal autonomic depression in ES. Despite a reduction in Total, LF, and HF power, the LF/HF ratio, which is a measure of sympatho-vagal balance, was elevated during the post-ictal period for ES. These results suggest that the relative reduction in HF (parasympathetic function) is more pronounced than the reduction in LF (baro-reflex function). In adjusted logistic regression models, SDNN, HF, LF, and TP are significantly lower during the post-ictal period following convulsive ES vs. convulsive FDS.

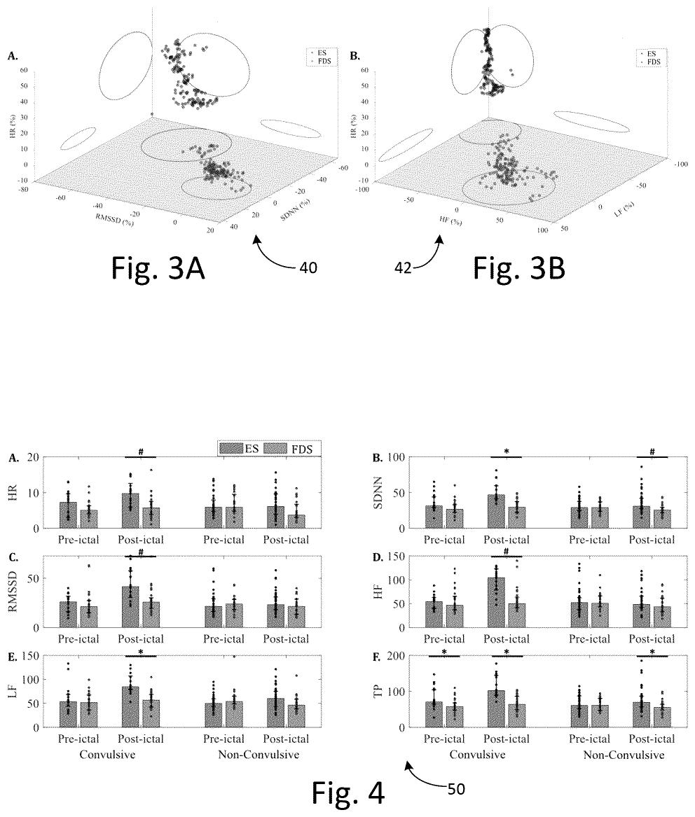

FIG. 3A is a graph 40 of convulsive ES group averages during every post-ictal epoch show an increase in heart rate and reductions in time domain and frequency domain. FIG. 3B is a graph 42 of convulsive ES group averages during every post-ictal epoch with autonomic measures compared to show an increase in heart rate and reductions in time domain and to FDS. Ellipses are centered at mean with radii 2 standard deviations.

When plotting multiple time (FIG. 3A) and frequency (FIG. 3B) domain HRV measures throughout the 150-minutes following the convulsive events on 3-dimensional plots, ES vs. FDS points are in different spatial regions. In addition to the individual points being in different non-overlapping regions in 3 dimensions, ellipses (mean±2SD) for ES vs. FDS plotted on the coordinate planes are distant and non-overlapping for each of the 2-dimensional comparisons. These results illustrate that accompanying the large HR increase in ES, there is marked autonomic withdrawal of total (SDNN), vagal (RMSSD, HF), and baroreflex (LF) function.

Next, we evaluated the variability in each HRV measure surrounding seizures. There is a significant difference in the relative variability (coefficient of variation: standard deviation divided by mean) of autonomic measures in ES vs. FDS (FIG. 4). While the level of autonomic function is depressed following an ES convulsive event, the relative variability in autonomic function increases during the 2.5-hour post-ictal period.

FIG. 4 is a series of graphs 50 of pre- and post-ictal coefficient of variation (100×standard deviation/mean). There is more relative variation in all post-ictal autonomic measures following a convulsive ES event than convulsive FDS (A-F). There is more relative variation in total autonomic function following non-convulsive ES events compared to FDS (B,F). Statistical significance: * p<0.05 in Wilcoxon's rank-sum test; #p<0.05 in Wilcoxon's rank-sum test and logistic regression adjusted for age, sex, and administration of lorazepam.

Based on the peri-ictal differences and variability in HR and autonomic function seen in ES vs. FDS, we performed classification modeling to evaluate whether these measures distinguish ES vs. FDS (Table 3).

TABLE 3
Non-Convulsive

| | ROC | Sensitivity | Specificity |
|---|---|---|---|
| Univariable Models | | | |
| Pre-ictal HR | 0.753 | 79.5 | 39.9 |
| | (0.673, 0.833) | (57.2, 99.9) | (1.4, 78.3) |
| Delta HR | 0.721 | 78.8 | 37.9 |
| | (0.656, 0.786) | (41.6, 99.9) | (0.1, 96.8) |
| Post-ictal HR | 0.780 | 85.4 | 50.5 |
| | (0.702, 0.858) | (60.9, 99.9) | (4.6, 96.3) |
| Multivariable Models | | | |
| Pre-ictal HR + Delta HR + | 0.751 | 75.7 | 38.7 |
| Post-ictal HR | (0.671, 0.831) | (42.5, 99.9) | (0.1, 84.4) |

Convulsive

| | ROC | Sensitivity | Specificity |
|---|---|---|---|
| Univariable Models | | | |
| Delta HR | 0.977 | 88.3 | 91.5 |
| | (0.955, 0.999) | (55.5, 99.9) | (71.6, 99.9) |
| Post-ictal HR | 0.962 | 81.6 | 85.6 |
| | (0.935, 0.989) | (45.2, 99.9) | (58.8, 99.9) |
| Post-ictal SDNN | 0.871 | 80.5 | 85.0 |
| | (0.798, 0.944) | (41.6, 99.9) | (59.5, 99.9) |
| Post-ictal RMSSD | 0.855 | 65.0 | 84.2 |
| | (0.775, 0.935) | (17.8, 99.9) | (58.3, 99.9) |
| Post-ictal HF Power | 0.932 | 88.3 | 84.1 |
| | (0.879, 0.985) | (58.1, 99.9) | (58.4, 99.9) |
| Post-ictal LF Power | 0.941 | 82.9 | 89.3 |
| | (0.892, 0.990) | (48.9, 99.9) | (65.9, 99.9) |
| Post-ictal Total Power | 0.893 | 86.3 | 83.0 |
| | (0.830, 0.956) | (51.4, 99.9) | (53.9, 99.9) |
| Post-ictal SDNN COV | 0.882 | 70.6 | 82.5 |
| | (0.823, 0.941) | (24.2, 99.9) | (55.1, 99.9) |
| Post-ictal HF Power COV | 0.868 | 68.4 | 89.5 |
| | (0.797, 0.939) | (24.3, 99.9) | (65.0, 99.9) |
| Post-ictal LF Power COV | 0.911 | 77.8 | 87.9 |
| | (0.852, 0.970) | (35.8, 99.9) | (60.6, 99.9) |
| Post-ictal Total Power COV | 0.892 | 68.0 | 80.7 |
| | (0.835, 0.949) | (19.1, 99.9) | (48.5, 99.9) |
| Multivariable Models | | | |
| Delta + Post-ictal HR | 0.990 | 92.3 | 93.6 |
| | (0.976, 0.999) | (63.5, 99.9) | (72.3, 99.9) |
| Post-ictal HR + SDNN + | 0.967 | 76.9 | 87.4 |
| RMSSD | (0.934, 0.999) | (36.5, 99.9) | (61.4, 99.9) |
| Post-ictal HR + HF | 0.980 | 79.6 | 92.3 |
| Power + LF Power | (0.956, 0.999) | (39.3, 99.9) | (71.5, 99.9) |
| Post-ictal HR + HR COV | 0.971 | 83.7 | 89.4 |
| | (0.944, 0.998) | (49.9, 99.9) | (62.6, 99.9) |
| Post-ictal SDNN + SDNN | 0.968 | 83.4 | 90.6 |
| COV | (0.939, 0.997) | (47.4, 99.9) | (66.0, 99.9) |
| Post-ictal RMSSD + | 0.899 | 71.2 | 87.9 |
| RMSSD COV | (0.826, 0.972) | (28.0, 99.9) | (64.8, 99.9) |
| Post-ictal HF Power + HF | 0.945 | 86.6 | 90.2 |
| Power COV | (0.894, 0.996) | (53.2, 99.9) | (67.3, 99.9) |
| Post-ictal LF Power + LF | 0.975 | 85.4 | 92.3 |
| Power COV | (0.942, 0.999) | (53.0, 99.9) | (73.0, 99.9) |
| Post-ictal Total Power + | 0.975 | 91.5 | 91.7 |
| Total Power COV | (0.950, 0.999) | (60.9, 99.9) | (72.6, 99.9) |

Table 3 illustrates pre-ictal, pre-to-post-ictal delta, and post-ictal heart rate distinguish non-convulsive ES vs. non-convulsive FDS. Pre-to-post-ictal heart rate delta, and post-ictal heart rate and autonomic measures distinguish convulsive ES vs. convulsive FDS events with high sensitivity, specificity, and area under the ROC curve. Mean and 95% confidence intervals were generated from 1000 iterations on randomly generated 75% training, 25% testing groups. Convulsive and non-convulsive events were analyzed separately. All HRV abbreviations and autonomic correlates are defined in Table 1 above.

HR distinguishes non-convulsive ES vs. non-convulsive FDS with an ROC>0.7 and sensitivity ≥79%, but its clinical diagnostic utility is limited as specificity is ≤50%. The best performing model had an optimal threshold of >−1.3% post-ictal change. For convulsive events, Delta and post-ictal HR have the highest sensitivity and specificity with ROC of 0.98 (95% CI, 0.96-1) and 0.96 (95% CI, 0.94-0.99), respectively. The optimal cut-off threshold to distinguish convulsive ES vs. convulsive FDS is a 19.5% (95% CI, 11.7-27.3) pre-to-post-ictal increase in HR across the event.

Consistent with the large differences in autonomic measures for convulsive ES vs. convulsive FDS, the post-ictal SDNN, RMSSD, LF, HF, and TP each have ROC>0.85. Optimal diagnostic cut-off thresholds for each measure are presented in Table 4. Similarly, post-ictal coefficient of variation for SDNN, HF, LF, and TP each have an ROC>0.86.

TABLE 4
Classification Model Thresholds

| | Mean Threshold | >Threshold | |
|---|---|---|---|
| | (% Change) | Predicts | 95% CI |
| HRV Timepoint | | | |
| (Non-Convulsive) | | | |
| Pre-ictal HR | 2.5 | FDS | (−2.2, 7.1) |
| HR Delta | −1.8 | ES | (−14.4, 10.8) |
| Post-ictal HR | −1.3 | ES | (−26.0, 23.3) |
| HRV Timepoint | | | |
| (Convulsive) | | | |
| HR Delta | 19.5 | ES | (11.7, 27.3) |
| Post-ictal HR | 17.3 | ES | (7.5, 27.0) |
| Post-ictal SDNN | −30.4 | FDS | (−38.4, −22.4) |
| Post-ictal RMSSD | −32.8 | FDS | (−53.8, −11.8) |
| Post-ictal HF Power | −76.5 | FDS | (−88.2, −64.8) |
| Post-ictal LF Power | −64.5 | FDS | (−87.7, −41.2) |
| Post-ictal Total Power | −52.4 | FDS | (−61.4, −43.4) |

Table 4 illustrates the cut-off thresholds determined by the univariable classification models presented in Table 3. Threshold units are within-subject percent change from the baseline period 150-60 minutes before event onset. Mean and 95% confidence intervals were determined over 1000 iterations with randomly selected 75% training groups. Non-convulsive events with a % change in HR greater than the threshold leading up to the event (preictal) were predicted to be FDS. Non-convulsive events with a % change in HR greater than the threshold during the delta and post-ictal periods were predicted to be ES. Convulsive events with a % change in HR greater than the threshold were predicted to be ES, while convulsive events with a % change in HRV measures less than the threshold (greater in magnitude) were predicted to be ES.

The combination of several HRV measures in a multi-variable model strengthens the accuracy of the classification for convulsive ES vs. convulsive FDS. Combining delta and post-ictal HR in the same classification model yields 92% sensitivity, 94% specificity, with 0.99 ROC. The combination of post-ictal HR with time-domain or frequency domain HRV measures increases the ROC compared to the univariable models. Likewise, combining each post-ictal HRV measure with its corresponding coefficient of variation yields ROC>0.89 in all measures.

ECG 14 recordings can occur for, in one embodiment, full 150 min pre-ictal and 150 min post-ictal periods (e.g., lead displacement or medical intervention). Thus, we determined whether there is a minimum duration of ECG recording needed, and if there are specific timepoints that provide the best distinction between ES and FDS.

FIG. 5 is a series of graphs of the temporal dependence of heart rate and HRV in distinguishing convulsive ES vs. convulsive FDS. FIG. 5, graph A shows1000 iterations (random 75% training, 25% testing) of logistic regression models were run on: 10-minute sliding epochs; epochs starting at the event and growing away from the event; epochs starting 150-minutes from the event and growing toward the event; and pre-to-post-ictal Delta of growing size. FIG. 5, graphs B-H are plots of mean ROC at each timepoint with 95% confidence bands (shaded) over the 1000 iterations. Horizontal dashed lines represent an ROC of 0.7. All HRV abbreviations and autonomic correlates are defined in Table 1 above. We assessed (1) each 10-minute sliding epoch (1-minute steps), (2) epochs that increased in duration outward from the seizure start/end (3) epochs that started 150 min away from the event and increased in size towards the event, and (4) the pre-ictal to post-ictal difference (delta) where the size of the period grew in duration away from the event (FIG. 5, graph A). During the post-ictal period, regardless of the timepoint and period duration, HR, total autonomic (SDNN & TP), vagal (RMSSD & HF), and baroreflex (LF) function all distinguished convulsive ES vs. convulsive FDS with an ROC>0.7 (FIG. 5, graphs B-H). As a potential easy-to-apply clinical tool, HR surrounding a convulsive seizure provides an ES vs. FDS diagnostic marker. Thus, regardless of the timepoint or size of the recording period, post-ictal HR and HRV measures distinguish convulsive ES vs. convulsive FDS.

The present invention accordingly provides a comprehensive evaluation of HR and autonomic function in ES and FDS patients. The present invention provides markers that distinguish convulsive ES vs. convulsive FDS events. In contrast to examining HR and autonomic function during small static and often heterogenous peri-ictal periods, one can map the temporal evolution of these measures in the 5 h surrounding seizures. There are large pre-to-post-ictal changes in HR and HRV measures surrounding both convulsive and non-convulsive ES, but not FDS. Despite HR being different surrounding non-convulsive ES vs. non-convulsive FDS, the clinical utility is limited due to poor specificity. In contrast, HR and HRV measures individually and collectively distinguish convulsive ES vs. convulsive FDS with a high level of sensitivity and specificity. Thus, ECG 14-derived autonomic markers may represent a novel non-invasive tool to aid in distinguishing ES vs. FDS. These results open the possibility for future out-of-hospital wearable sensors to evaluate the peri-ictal changes in HR and HRV measures, which may aid in the clinical diagnosis of ES vs. FDS in out-of-hospital settings.

One advantage of the present invention is that many people have limited geographical and financial access to emergency medical unites (EMU) and evaluation by an epileptologist. Also, habitual events may not be captured in the EMU. While several semiological manifestations are associated with ES and FDS, none are pathognomonic. Neuroimaging measures, accelerometry, HR, autonomic function, as well as immune-associated protein, hormonal, and enzyme levels differ in ES vs. FDS Yet, they do not accurately identify all ES events. While several clinical features are typically associated with both ES and FDS, most do not reliably distinguish ES vs. FDS.

Video data can yield an 86-89% diagnostic accuracy for ES and FDS. Similar to our study, when stratifying for (non-) convulsive events, the high accuracy of video was only seen for convulsive events. Serum levels of immune response-associated proteins and HR above 130 bpm distinguish ES vs. FDS. Ictal cardiosympathetic index distinguishes non-convulsive ES vs. non-convulsive FDS with high accuracy. A small case series using a multi-parameter recordings during ictus yielded 78% predictive accuracy for ES vs. FDS. In contrast to using ictal timepoints that are often contaminated by muscle artifact, the present model uses pre-ictal and post-ictal timepoints when the signal is usually cleaner. Unlike small static ictal or inter-ictal timepoints, our analytical biomarker can be applied to ECGs recordings throughout the 150 min post-ictal period.

Diagnostic tools to collect the above information are not as ubiquitous as ECG recordings and therefore may pose barriers to implementation. In contrast to video, EEG, and blood-based diagnostic biomarkers, ECGs 14 are ubiquitously available and thus may provide an out-of-hospital diagnostic tool. This potential ES vs. FDS diagnostic biomarker will improve geographic and financial access to medical testing, and capture events in the person's natural environment. This will direct people to the appropriate health care provider and accelerate the time to the initiation of medical therapy.

The present state of knowledge regarding the autonomic changes surrounding ES and FDS is limited. These differences may be related to heterogeneous physiological states leading up to the seizure (e.g., awake vs. asleep). Within the same study and between studies, there are heterogeneities in the durations of the recordings used for analyses, and many periods are below the durations recommended for HRV analysis. Other contributors may include small cohorts, varying numbers of events from the same individual, as well as inconsistent study protocols and analytical approaches.

Accordingly, the present study employed a rigorous and comprehensive analysis protocol. Each participants' peri-ictal HRV measures were normalized to their own pre-ictal baseline, which accounts for both inter- and intra-patient variability. Unlike small static ictal or inter-ictal timepoints, our analytical biomarkers are applied to ECG 14 recordings throughout the 150-minute pre- and post-ictal periods. Analysis was run on overlapping 5-minute windows in the 2.5 h preceding event onset, and 2.5 h following event end, which reduces the bias of timepoint selection. This prospective study used a standardized recording protocol with one temporally isolated, habitual diagnostic event per participant, which eliminates potential bias from overweighting patients with multiple events. Strict inclusion criteria reduced potential bias of medications, devices, and comorbidities that affect HR and autonomic function. All analyses were performed following established HRV analysis guidelines.

There is great heterogeneity in the HR changes immediately preceding an ES. Bruno et al. (2018) reported that HR increased preceding ES in 36% of cases while pre-ictal HR reductions were rare. Similar to our results, other investigators reported that, despite a small increase in HR and SDNN leading up to FDS, pre-ictal measures do not differ for ES vs. FDS events. Yet, others in the art reported that pre-ictal and post-ictal HR differ for focal impaired awareness seizures vs. FDS events.

Previous studies reported increased HR during both ES and FDS, although more prevalent during ES than FDS, as well as a high prevalence of ictal tachycardia during ES events. According to Devinsky et al. (2011), a >30% increase in HR during the seizure predicts 97% of ES events. Opherk et al. (2002) demonstrated that ictal HR had an 83% sensitivity and 96% specificity for ES. Ponnusamy et al. 2012 reported that ictal cardiac sympathetic index distinguishes ES vs. FDS, and correctly identifies ES, but not FDS.

For convulsive events, post-ictal HR and autonomic measures significantly differ for ES vs. FDS. Similar to our results, others reported that in ES the post-ictal HR and autonomic changes last up to several hours, contrary to a quick return to baseline following FDS events. In contrast, other results do not demonstrate that these post-ictal changes distinguish convulsive ES vs. convulsive FDS. While yet others showed that FDS events are associated with post-ictal changes in HR, total autonomic and vagal function. The present study did not see differences compared to the baseline period. Thus, the absence of a significant and sustained change in HR and HRV measures during the post-ictal period distinguishes convulsive FDS from ES.

With reference again to FIG. 1 and the above teaching, it can be seen the invention provides a method for distinguishing the type of seizures in a human patient 12, by receiving HRV analytical data from a patient 12, the HRV data sent from an electrocardiogram device (ECG) 14, to a diagnostic analytical platform, and distinguishing if a predetermined type of seizure has occurred in the patient 12. The receipt of the patent data 8 and distinguishing the onset can occur at a remote diagnostic analytical platform 16 on can occur in a single, integrated device.

The distinguishing of the onset of a predetermined type of seizure in the patient 8 can be distinguishing one of: an epileptic seizure (ES), or a functional or dissociative seizure (FDS). The distinguishing of a predetermined type of seizure can be distinguishing an ES or FDS by the presence or absence of predetermined changes in the HRV analytical data. The method can further involve distinguishing predetermined types of seizures in a series of seizures within a predetermined duration within the patient 12, which can be, in one embodiment, a period of 1-10 hours. The method can further include creating a model for distinguishing the a predetermined type of seizure in the patient based upon received HRV data from the ECG 14. In one embodiment, the model is created using a logistic regression model.

As used herein, the term “diagnostic analytical platform” is a computer platform, or the hardware infrastructure on which software is executed. For example, in a single computer system, this would be the computer's architecture, operating system (OS), and runtime libraries. In the case of an application program, the most relevant layer is the OS, which can be called a platform itself, such as Unix, Linux, DOS, Windows, or MacOS. In a multi-computer system, such as in the case of offloading processing, it would encompass both the host computer's hardware, operating system (OS), and runtime libraries along with other computers utilized for processing that are accessed via application programming interfaces or a web browser. In hardware, examples of a computer platform are ARM architecture, PC architecture, ASRock, or AMD. In general, as long as it is a required component for a program code to execute, it is part of the computing platform.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A system for distinguishing a type of seizures in a human patient, comprising:
an electrocardiogram device (ECG) selectively recording and transmitting cardiac data from a patient, the ECG selectively providing heart rate variability (HRV) metrics for the patient; and
a diagnostic analytical platform that receives the HRV analytical metrics from the ECG, and the diagnostic analytical platform further configured to utilize an analytical diagnostic algorithm to distinguish that a predetermined type of seizure has occurred in the patient.

2. The system of claim 1, wherein the predetermined type of seizure is one of: an epileptic seizure (ES), or a functional or dissociative seizure (FDS).

3. The system of claim 1, wherein the ECG device is further configured to create HRV analytical data from cardiac data for the patient.

4. The system of claim 2, wherein an ES is distinguished by predetermined changes in the HRV analytical data over a predetermined duration.

5. The system of claim 2, wherein an FDS is distinguished by an absence of changes in HRV analytical data over a predetermined duration.

6. The system of claim 1, wherein the diagnostic analytical platform further configured to distinguish predetermined types of seizures in a series of seizures within a predetermined duration within the patient.

7. The system of claim 1, wherein the diagnostic analytical platform further configured to create a model for distinguishing the predetermined type of seizure in the patient.

8. The system of claim 7, wherein the model is a logistic regression model.

9. A method for distinguishing a type of seizures in a human patient, comprising:
receiving, at a diagnostic analytical platform, HRV analytical data for a patient, the HRV analytical data sent from an electrocardiogram device (ECG); and
distinguishing, on the diagnostic analytical platform, that a predetermined type of seizure occurred in the patient.

10. The method of claim 9, wherein distinguishing the predetermined type of seizure in the patient is distinguishing one of: an epileptic seizure (ES), or a functional or dissociative seizure (FDS).

11. The method of claim 9, further comprising creating, at the ECG, HRV analytical data from cardiac data for the patient.

12. The method of claim 10, wherein distinguishing the predetermined type of seizure is distinguishing predetermined changes in the HRV analytical data over a predetermined duration.

13. The method of claim 10, wherein distinguishing the predetermined type of seizure is distinguishing an FDS by an absence of predetermined changes in the HRV analytical data over a predetermined duration.

14. The method of claim 9, further comprising distinguishing predetermined types of seizures in a series of seizures within a predetermined duration within the patient.

15. The method of claim 9, further comprising creating a model, at the diagnostic analytical platform, for distinguishing the predetermined type of seizure that occurred in the patient

16. The method of claim 15, wherein creating a model is creating a logistic regression model.

17. A device for distinguishing a type of seizures in a human patient, comprising:
an electrocardiogram device (ECG) selectively recording and transmitting cardiac data from a patient, the ECG selectively providing heart rate variability (HRV) metrics for the patient; and
a diagnostic analytical platform that receives the HRV analytical metrics from the ECG, and the diagnostic analytical platform further configured to utilize an analytical diagnostic algorithm to distinguish that a predetermined type of seizure has occurred in the patient.

18. The device of claim 17, wherein the predetermined type of seizure is one of: an epileptic seizure (ES), or a functional or dissociative seizure (FDS).

19. The device of claim 17, wherein the diagnostic analytical platform further configured to distinguish predetermined types of seizures in a series of seizures within a predetermined duration within the patient.

20. The device of claim 17, wherein the diagnostic analytical platform further configured to create a model for distinguishing that a predetermined type of seizure occurred in the patient.
